SEQUENCE LISTING (14 sequences)

SEQ ID NO: 1 - DNA, 1791 bp, Neisseria meningitidis
atgcgctacaaaccccttctgcttgccctgatgctcgttttttccacgcccgccgttgccgcccacgacgcggcacacaaccgttccgccgaagtgaaaaaacagacgaagaacaaaaaagaacagcccgaagcggcggaaggcaaaaaagaaaaaggcaaaaatggcgcagtgaaagataaaaaaacaggcggcaaagaggcggcaaaagagggcaaagagtccaaaaaaaccgccaaaaaccgcaaagaagcagagaaggaggcgacatccaggcagtctgcgcgcaaaggacgcgaaggggataagaaatcgaaggcggaacacaaaaaggcacatggcaagcccgtgtccggatccaaagaaaaaaacgcaaaaacacagcctgaaaacaaacaaggcaaaaaagaggcaaaaggacagggcaatccgcgcaagggcggcaaggcggaaaaagacactgtttctgcaaataaaaaagtccgttccgacaagaacggcaaagcagtgaaacaggacaaaaaatacagggaagagaaaaatgccaaaaccgattccgacgaattgaaagccgccgttgccgctgccaccaatgatgtcgaaaacaaaaaagccctgctcaaacaaagcgaaggaatgctgcttcatgtcagcaattccctcaaacagcttcaggaagagcgtatccgccaagagcgtatccgtcaggcgcgcggcaaccttgcttccgtcaaccgcaaacagcgcgaggcttgggacaagttccaaaaactcaataccgagctgaaccgtttgaaaacggaagtcgccgctacgaaagcgcagatttcccgtttcgtatcggggaactataaaaacagccagccgaatgcggttgccctgttcctgaaaaacgccgaaccgggtcagaaaaaccgctttttgcgttatacgcgttatgtaaacgcctccaatcgggaagttgtcaaggatttggaaaaacagcagaaggctttggcggtacaagagcagaaaatcaacaatgagcttgcccgtttgaagaaaattcaggcaaacgtgcaatctctgctgaaaaaacagggtgtaaccgatgcggcggaacagacggaaagccgcagacagaatgccaaaatcgccaaagatgcccgaaaactgctggaacagaaagggaacgagcagcagctgaacaagctcttgagcaatttggagaagaaaaaggccgaacaccgcattcaggatgcggaagcaaaaagaaaattggctgaagccagactggcggcagccgaaaaagccagaaaagaagcggcgcagcagaaggctgaagcacgacgtgcggaaatgtccaacctgaccgccgaagacaggaacatccaagcgccttcggttatgggtatcggcagtgccgacggtttcagccgcatgcaaggacgtttgaaaaaaccggttgacggtgtgccgaccggacttttcgggcagaaccggagcggcggcgatatttggaaaggcgtgttctattccactgcaccggcaacggttgaaagcattgcgccgggaacggtaagctatgcggacgagttggacggctacggcaaagtggtcgtggtcgatcacggcgagaactacatcagcatctatgccggtttgagcgaaatttccgtcggcaagggttatatggtcgcggcaggaagcaaaatcggctcgagcgggtcgctgccggacggggaagaggggctttacctgcaaatacgttatcaaggtcaggtattgaacccttcgagctggatacgttga

SEQ ID NO: 2 - protein, 596 aa, Neisseria meningitidis
MRYKPLLLALMLVFSTPAVAAHDAAHNRSAEVKKQTKNKKEQPEAAEGKKEKGKNGAVKDKKTGGKEAAKEGKESKKTAKNRKEAEKEATSRQSARKGREGDKKSKAEHKKAHGKPVSGSKEKNAKTQPENKQGKKEAKGQGNPRKGGKAEKDTVSANKKVRSDKNGKAVKQDKKYREEKNAKTDSDELKAAVAAATNDVENKKALLKQSEGMLLHVSNSLKQLQEERIRQERIRQARGNLASVNRKQREAWDKFQKLNTELNRLKTEVAATKAQISRFVSGNYKNSQPNAVALFLKNAEPGQKNRFLRYTRYVNASNREVVKDLEKQQKALAVQEQKINNELARLKKIQANVQSLLKKQGVTDAAEQTESRRQNAKIAKDARKLLEQKGNEQQLNKLLSNLEKKKAEHRIQDAEAKRKLAEARLAAAEKARKEAAQQKAEARRAEMSNLTAEDRNIQAPSVMGIGSADGFSRMQGRLKKPVDGVPTGLFGQNRSGGDIWKGVFYSTAPATVESIAPGTVSYADELDGYGKVVVVDHGENYISIYAGLSEISVGKGYMVAAGSKIGSSGSLPDGEEGLYLQIRYQGQVLNPSSWIR

SEQ ID NO: 3 - DNA, 1131 bp, Neisseria meningitidis
atggcgttttgcaccagtttgggagtgatgatggaaacacagctttacatcggcatcatgtcgggaaccagcatggacggggcggatgccgtactgatacggatggacggcggcaaatggctgggcgcggaagggcacgcctttaccccctaccccggcaggttacgccgccaattgctggatttgcaggacacaggcgcagacgaactgcaccgcagcaggattttgtcgcaagaactcagccgcctatatgcgcaaaccgccgccgaactgctgtgcagtcaaaacctcgcaccgtccgacattaccgccctcggctgccacgggcaaaccgtccgacacgcgccggaacacggttacagcatacagcttgccgatttgccgctgctggcggaacggacgcggatttttaccgtcggcgacttccgcagccgcgaccttgcggccggcggacaaggcgcgccactcgtccccgcctttcacgaagccctgttccgcgacaacagggaaacacgcgcggtactgaacatcggcgggattgccaacatcagcgtactcccccccgacgcacccgccttcggcttcgacacagggccgggcaatatgctgatggacgcgtggacgcaggcacactggcagcttccttacgacaaaaacggtgcaaaggcggcacaaggcaacatattgccgcaactgctcgacaggctgctcgcccacccgtatttcgcacaaccccaccctaaaagcacggggcgcgaactgtttgccctaaattggctcgaaacctaccttgacggcggcgaaaaccgatacgacgtattgcggacgctttcccgttttaccgcgcaaaccgtttgcgacgccgtctcacacgcagcggcagatgcccgtcaaatgtacatttgcggcggcggcatccgcaatcctgttttaatggcggatttggcagaatgtttcggcacacgcgtttccctgcacagcaccgccgacctgaacctcgatccgcaatgggtggaagccgccgcatttgcgtggttggcggcgtgttggattaatcgcattcccggtagtccgcacaaagcaaccggcgcatccaaaccgtgtattctgggcgcgggatattattattga

SEQ ID NO: 4 - protein, 376 aa, Neisseria meningitidis
MAFCTSLGVMMETQLYIGIMSGTSMDGADAVLIRMDGGKWLGAEGHAFTPYPGRLRRQLLDLQDTGADELHRSRILSQELSRLYAQTAAELLCSQNLAPSDITALGCHGQTVRHAPEHGYSIQLADLPLLAERTRIFTVGDFRSRDLAAGGQGAPLVPAFHEALFRDNRETRAVLNIGGIANISVLPPDAPAFGFDTGPGNMLMDAWTQAHWQLPYDKNGAKAAQGNILPQLLDRLLAHPYFAQPHPKSTGRELFALNWLETYLDGGENRYDVLRTLSRFTAQTVCDAVSHAAADARQMYICGGGIRNPVLMADLAECFGTRVSLHSTADLNLDPQWVEAAAFAWLAACWINRIPGSPHKATGASKPCILGAGYYY

SEQ ID NO: 5 - DNA, 1857 bp, Neisseria meningitidis
atgttgaacaaaatattttcctggttcgagtcccgaatcgacccttatcccgaagccgccccgaaaacgccagaaaaaggcttgtggcggtttgtctggagcagcatggccggcgtgcggaaatggatagccgccctggctgcgctgaccgccggcatcggcattatggaagccctggtttttcaatttatgggcaaaatcgtggagtggctcggcaaatacgcgcccgccgaactgtttgccgaaaaaagttgggaactggcggcaatggcggcgatgatggtattttcggttgcgtgggcgtttgccgcgtccaacgtgcgcctgcaaacccttcagggcgtgttccccatgcgcctgcgctggaacttccaccgcctgatgctgaaccaaagcctcggtttttatcaggacgaatttgccggacgcgtgtccgccaaagtcatgcagaccgcgctggcgttgcgcgacgcggtgatgacggttgccgatatggtcgtttatgtgtcggtgtatttcattacctccggcgtgattctcgcctcgctcgactcatggctgctgctgccctttatcggctggattgtcggtttcgcttcggtgatgcgcctgctgattcccaaattggggcaaaccgccgcatggcaggcggatgcccgctcgctgatgaccggccgcattaccgatgcctattccaatatcgccaccgtcaaactcttctcccacggcgcgcgtgaagccgcctatgccaagcagtcgatggaagaatttatggttacggtgcgcgcccaaatgcggctggcgacgctgctgcattcgtgcagcttcatcgtcaacacctccctgaccctctccaccgccgcactgggcatctggctctggcacaacgggcaggtcggcgtgggcgcggttgctacagccaccgccatggcgttgcgcgtcaacggtttgtcgcaatacattatgtgggaatccgcgcggctgtttgaaaacatcggcaccgtcggcgacggcatggcaaccctgtccaaaccgcacaccatcctcgacaagccccgggcactgccgctgaacgtgccgcaaggcgcaatcaaatttgaacacgtcgatttctcctacgaagcgggcaaaccgctgctcaacggcttcaacctcaccatccgcccgggcgaaaaagtcggcttgatcggacgcagcggcgcgggcaaatccaccatcgtcaacctgcttttgcgcttctacgaaccgcaaagcggcacggtttcgatcgacgggcaggacataagcggcgttacccaagaatctttacgcgcccaaatcggtttggtcacgcaagatacctcgctgctgcaccgttccgtgcgcgacaacattatttacggccgccccgacgcgaccgatgccgaaatggtttctgccgccgaacgcgccgaagccgccggcttcatccccgacctttccgatgccaaagggcggcgcggctacgacgcacacgtcggcgaacgcggcgtgaaactctccggcgggcaacgccagcgcatcgccatcgcccgcgtgatgctcaaagacgcaccgattcttcttttggacgaagccaccagcgcgctcgattccgaagtcgaagccgccatccaagaaagcctcgacaaaatgatggacggcaaaaccgtcatcgccatcgcccaccgcctctccaccatcgccgcaatggacaggctcgtcgtcctcgacaaaggccgcatcatcgaagaaggcacacacgccgaactcctcgaaaaacgcgggctttacgccaaactctgggcgcaccagagcggcggcttcctcaacgaacacgtcgagtggcagcacgactga

SEQ ID NO: 6 - protein, 618 aa, Neisseria meningitidis
MLNKIFSWFESRIDPYPEAAPKTPEKGLWRFVWSSMAGVRKWIAALAALTAGIGIMEALVFQFMGKIVEWLGKYAPAELFAEKSWELAAMAAMMVFSVAWAFAASNVRLQTLQGVFPMRLRWNFHRLMLNQSLGFYQDEFAGRVSAKVMQTALALRDAVMTVADMVVYVSVYFITSGVILASLDSWLLLPFIGWIVGFASVMRLLIPKLGQTAAWQADARSLMTGRITDAYSNIATVKLFSHGAREAAYAKQSMEEFMVTVRAQMRLATLLHSCSFIVNTSLTLSTAALGIWLWHNGQVGVGAVATATAMALRVNGLSQYIMWESARLFENIGTVGDGMATLSKPHTILDKPRALPLNVPQGAIKFEHVDFSYEAGKPLLNGFNLTIRPGEKVGLIGRSGAGKSTIVNLLLRFYEPQSGTVSIDGQDISGVTQESLRAQIGLVTQDTSLLHRSVRDNIIYGRPDATDAEMVSAAERAEAAGFIPDLSDAKGRRGYDAHVGERGVKLSGGQRQRIAIARVMLKDAPILLLDEATSALDSEVEAAIQESLDKMMDGKTVIAIAHRLSTIAAMDRLVVLDKGRIIEEGTHAELLEKRGLYAKLWAHQSGGFLNEHVEWQHD

SEQ ID NO: 7 - DNA, 1410 bp, Neisseria meningitidis
atgacagcacaaaccctctacgacaaactttggaacagccacgtcgtccgcgaagaagaagacggcaccgtcctgctctacatcgaccgccatttggtgcacgaagttaccagccctcaggcatttgaaggcttgaaaatggcggggcgcaagctgtggcgcatcgacagcgtcgtctccaccgccgaccacaacaccccgaccggcgattgggacaaaggcatccaagacccgatttccaagctgcaagtcgatactttggacaaaaacattaaagagtttggcgcactcgcctattttccgtttatggacaaaggtcagggcatcgtacacgttatgggccccgaacaaggcgcgaccctgcccggtatgaccgtcgtctgcggcgactcgcacacttccacccacggcgcattcggcgcactggcgcacggcatcggcacttccgaagtcgagcacaccatggcgacccaatgtattaccgcgaaaaaatccaaatccatgctgatttccgttgacggcaaattaaaagcgggcgttaccgccaaagacgtggcgctctacatcatcgggcaaatcggcacggcaggcggtacaggctacgccatcgagtttggcggcgaagccatccgcagcctttctatggaaagccgcatgactttatgcaatatggcgattgaggcaggcgcgcgctcaggcatggttgccgtcgaccaaaccaccatcgactacgtaaaagataaacccttcgcacccgaaggcgaagcgtgggacaaagccgtcgagtactggcgtacgctggtgtctgacgaaggtgcggtattcgacaaagaataccgtttcaacgccgaagacatcgaaccgcaagtcacttggggtacctcgcctgaaatggttttagacatcagcagcaaagtgccgaatcctgccgaagaaaccgatccggtcaaacgcagcggtatggaacgcgcccttgaatacatgggcttggaagccggtacgccattaaacgaaatccccgtcgacatcgtattcatcggctcttgcaccaacagccgcatcgaagacttgcgcgaagccgccgccatcgccaaagaccgcaaaaaagccgccaacgtacagcgcgtgttaatcgtccccggctccggtttggttaaagaacaagccgaaaaagaaggcttggacaaaattttcatcgaagccggttttgaatggcgcgaaccgggctgttcgatgtgtctcgccatgaacgccgaccgcctgaccccggggcaacgctgcgcctccacctccaaccgtaactttgaaggccgtcaaggcaacggcggacgtacccacctcgtcagccccgctatggcagcagccgccgccgttaccggccgctttaccgacatccgcatgatggcgtaa

SEQ ID NO: 8 - protein, 469 aa, Neisseria meningitidis
MTAQTLYDKLWNSHVVREEEDGTVLLYIDRHLVHEVTSPQAFEGLKMAGRKLWRIDSVVSTADHNTPTGDWDKGIQDPISKLQVDTLDKNIKEFGALAYFPFMDKGQGIVHVMGPEQGATLPGMTVVCGDSHTSTHGAFGALAHGIGTSEVEHTMATQCITAKKSKSMLISVDGKLKAGVTAKDVALYIIGQIGTAGGTGYAIEFGGEAIRSLSMESRMTLCNMAIEAGARSGMVAVDQTTIDYVKDKPFAPEGEAWDKAVEYWRTLVSDEGAVFDKEYRFNAEDIEPQVTWGTSPEMVLDISSKVPNPAEETDPVKRSGMERALEYMGLEAGTPLNEIPVDIVFIGSCTNSRIEDLREAAAIAKDRKKAANVQRVLIVPGSGLVKEQAEKEGLDKIFIEAGFEWREPGCSMCLAMNADRLTPGQRCASTSNRNFEGRQGNGGRTHLVSPAMAAAAAVTGRFTDIRMMA

SEQ ID NO: 9 - DNA, 402 bp, Neisseria meningitidis
atgaaagacaagcacgattcttccgccatgcggctggacaaatggctttgggcggcacgttttttcaagacccgttcccttgcgcaaaagcacatcgaactgggtagggttcaagtaaacggctcgaaggtcaaaaacagtaaaaccatagacatcggcgatattatcgacctgacgctcaattcccttccctataaaatcaaggttaaaggtttgaaccaccaacgccgcccggcatccgaggcgcggcttctgtatgaagaggacgcgaaaacggcaacattgagggaagagcgcaaacagctcgaccaattcagccgcatcacttccgcctatcccgacggcagaccgaccaagcgcgaccgccgccaactggacaggctgaaaaaaggagactggtaa

SEQ ID NO: 10 - protein, 133 aa, Neisseria meningitidis
MKDKHDSSAMRLDKWLWAARFFKTRSLAQKHIELGRVQVNGSKVKNSKTIDIGDIIDLTLNSLPYKIKVKGLNHQRRPASEARLLYEEDAKTATLREERKQLDQFSRITSAYPDGRPTKRDRRQLDRLKKGDW

SEQ ID NO: 11 - DNA, 1011 bp, Neisseria meningitidis
atgaaccacaccgttaccctgcccgaccaaaccacctttgccgccaacgacggcgaaaccgttttgaccgctgccgcccgtcaaaacctcaacctgccccattcctgcaaaagcggtgtctgcggacaatgcaaagccgaactggtcagcggcgatattcaaatgggcggacactcggaacaggctttatccgaagcagaaaaagcgcaaggcaagattttgatgtgctgcaccactgcgcaaagcgatatcaacatcaacatccccggctacaaagccgatgccctacccgtccgcaccctgcccgcacgcatcgaaagtattattttcaaacacgatgtcgccctcctgaaacttgccctgcccaaagccccgccgtttgccttctacgccgggcaatacattgatttactgctgccgggcaacgtcagccgcagctactccatcgccaatttacccgaccaagaaggcattttggaactgcacatccgcaggcacgaaaacggtgtctgctcggaaatgattttcggcagcgaacccaaagtcaaagaaaaaggcatcgtccgcgttaaaggcccgctcggttcgtttaccttgcaggaagacagcggcaaacccgtcatcctgctggcaaccggcacaggctacgcccccatccgcagcatcctgctcgaccttatccgccaaggcagcaaccgcgccgtccatttctactggggcgcgcgtcatcaggatgatttgtatgccctcgaagaagcacaagggttggcatgccgtctgaaaaacgcctgcttcacccccgtattgtcccgccccggagagggctggcagggaagaaatggtcacgtacaagacatcgcggcacaagaccaccccgacctgtcggaatacgaagtatttgcctgcggttctccggccatgaccgaacaaacaaagaatctgtttgtgcaacagcataagctgccggaaaacttgtttttctccgacgcattcacgccgtccgcatcataa

SEQ ID NO: 12 - protein, 336 aa, Neisseria meningitidis
MNHTVTLPDQTTFAANDGETVLTAAARQNLNLPHSCKSGVCGQCKAELVSGDIQMGGHSEQALSEAEKAQGKILMCCTTAQSDININIPGYKADALPVRTLPARIESIIFKHDVALLKLALPKAPPFAFYAGQYIDLLLPGNVSRSYSIANLPDQEGILELHIRRHENGVCSEMIFGSEPKVKEKGIVRVKGPLGSFTLQEDSGKPVILLATGTGYAPIRSILLDLIRQGSNRAVHFYWGARHQDDLYALEEAQGLACRLKNACFTPVLSRPGEGWQGRNGHVQDIAAQDHPDLSEYEVFACGSPAMTEQTKNLFVQQHKLPENLFFSDAFTPSAS

SEQ ID NO: 13 - DNA, 402 bp, Neisseria meningitidis; identical to SEQ ID NO: 9

SEQ ID NO: 14 - protein, 133 aa, Neisseria meningitidis; identical to SEQ ID NO: 10

(19) United States
(12) Patent Application Publication
(10) Pub. No.: US 2009/0226479 A1
(43) Pub. Date: Sep. 10, 2009

(54) VACCINES AND THEIR USE

(75) Inventors: Christoph Marcel Tang, London (GB); Yanwen Li, London (GB)

Correspondence Address: SHELDON MAK ROSE & ANDERSON PC, 100 Corson Street, Third Floor, PASADENA, CA, 91103-3842, US

(73) Assignee: IMPERIAL INNOVATIONS LIMITED, London (GB)

(21) Appl. No.: 12/158,919

(22) PCT Filed: Dec. 21, 2006
(86) PCT No.: PCT/GB06/04877

§ 371 Date: Aug. 29, 2008

(30) Foreign Application Priority Data
Dec. 23, 2005 (GB) PCT/GB2005/005113

(51) Int. Cl. A61K 39/02; C07K 14/22; C07H 21/04; A61K 38/16; A61K 39/12

(52) U.S. Cl. 424/190.1; 530/350; 536/23.7; 514/12; 424/204.1

(57) ABSTRACT

Various polypeptides, or a variant or fragment thereof or a fusion of these are described which are useful in a vaccine. The polypeptide may be a polypeptide comprising the amino acid sequence selected from any one of SEQ ID Nos 2, 4, 6, 8, 10, 12, 14;

or a fragment or variant thereof or a fusion of such a fragment or variant, and is useful in a vaccine against Neisseria meningitidis.

The present invention relates to vaccines and their use, and in particular to vaccines for meningococcal disease.

The listing or discussion of a prior-published document in this specification should not necessarily be taken as an acknowledgement that the document is part of the state of the art or is common general knowledge. The documents listed in the specification are hereby incorporated by reference.

Microbial infections remain a serious risk to human and animal health, particularly in light of the fact that many pathogenic microorganisms, particularly bacteria, are or may become resistant to anti-microbial agents such as antibiotics.

Vaccination provides an alternative approach to combating microbial infections, but it is often difficult to identify suitable immunogens for use in vaccines which are safe and which are effective against a range of different isolates of a pathogenic microorganism, particular a genetically diverse microorganism. Although it is possible to develop vaccines which use as the immunogen substantially intact microorganisms, such as live attenuated bacteria which typically contain one or mutations in a virulence-determining gene, not all microorganisms are amenable to this approach, and it is not always desirable to adopt this approach for a particular microorganism where safety cannot always be guaranteed. Also, some microorganisms express molecules which mimic host proteins, and these are undesirable in a vaccine.

A particular group of microorganisms for which it is important to develop further vaccines is Neisseria meningitidis which causes meningococcal disease, a life threatening infection which in the Europe, North America, developing countries and elsewhere remains an important cause of childhood mortality despite the introduction of the conjugate serogroup C polysaccharide vaccine. This is because infections caused by serogroup B strains (NmB), which express an α2-8 linked polysialic acid capsule, are still prevalent. The term “serogroup” in relation to N. meningitidis refers to the polysaccharide capsule expressed on the bacterium. The common serogroup in the UK causing disease is B, while in Africa it is A. Meningococcal septicaemia continues to carry a high case fatality rate; and survivors are often left with major psychological and/or physical disability. After a non-specific prodromal illness, meningococcal septicaemia can present as a fulminant disease that is refractory to appropriate anti-microbial therapy and full supportive measures. Therefore, the best approach to combating the public health menace of meningococcal disease is through prophylactic vaccination.

The non-specific early clinical signs and fulminant course of meningococcal infection mean that therapy is often ineffective. Therefore vaccination is considered the most effective strategy to diminish the global disease burden caused by this pathogen (Feavers (2000) ABC of meningococcal diversity. Nature 404, 451-2). Existing vaccines to prevent serogroup A, C, W135, and Y N. meningitidis infections are based on the polysaccharide capsule located on the surface of bacterium (Anderson et al (1994) Safety and immunogenicity of meningococcal A and C polysaccharide conjugate vaccine in adults. Infect Immun. 62, 3391-33955; Leach et al (1997) Induction of immunologic memory in Gambian children by vaccination in infancy with a group A plus group C meningococcal polysaccharide-protein conjugate vaccine. J Infect Dis. 175, 200-4; Lieberman et al (1996). Safety and immunogenicity of a serogroups A/C Neisseria meningitidis oligosaccharide-protein conjugate vaccine in young children. A randomized controlled trial. J. American Med. Assoc. 275, 1499-1503). Progress toward a vaccine against serogroup B infections has been more difficult as its capsule, a homopolymer of E2-8 linked sialic acid, is a relatively poor immunogen in humans. This is because it shares epitopes expressed on a human cell adhesion molecule, N-CAM1 (Finne et al (1983) Antigenic similarities between brain components and bacteria causing meningitis. Implications for vaccine development and pathogenesis. Lancet 2, 355-357). Indeed, generating immune responses against the serogroup B capsule might actually prove harmful. Thus, there remains a need for new vaccines to prevent serogroup B N. meningitidis infections.

The most validated immunologic correlate of protection against meningococcal disease is the serum bactericidal assay (SBA). The SBA evaluates the ability of antibodies (usually IgG2a subclass) in serum to mediate complement deposition on the bacterial cell surface, assembly of the membrane attack complex, and bacterial lysis. In the SBA, a known number of bacteria are exposed serial dilutions of the sera with a defined complement source. The number of surviving bacteria is determined, and the SBA is defined as the reciprocal of the highest dilution of serum that mediates 50% killing. The SBA is predictive of protection against serogroup C infections, and has been widely used as a surrogate for immunity against NmB infections. Importantly the SBA is a ready marker of immunity for the pre-clinical assessment of vaccines, and provides a suitable endpoint in clinical trials.

Most efforts at NmB vaccine development are directed toward defining effective protein subunits. There has been a major investment in ‘Reverse vaccinology’, in which genome sequences are interrogated for potentially surface expressed proteins which are expressed as heterologous antigens and tested for their ability to generate meaningful responses in animals. However, this approach is limited by 1) the computer algorithms for predicting surface expressed antigens, 2) failure to express many of potential immunogens, and 3) the total reliance on murine immune responses.

The key to a successful vaccine is to define antigen(s) that elicit protection against a broad range of disease isolates irrespective of serogroup or clonal group. A genetic screening method (which we have termed Genetic Screening for Immunogens or GSI) was used to isolate antigens that are conserved across the genetic diversity of microbial strains and this is exemplified in relation to meningococcal strains. This was done by identifying microbial antigens, such as N. meningitidis antigens, by GSI as described in more detail below; and validated by assessing the function of the immune response elicited by the recombinant antigens and by evaluating the protective efficacy of antigens (see Examples and see PCT/GB2004/005441 (published as WO 2005/060995 on 7 Jul. 2005) incorporated herein by reference). In essence, the GSI method relates to a method for identifying a polypeptide of a microorganism which polypeptide is associated with an immune response in an animal which has been subjected to the microorganism, the method comprising the steps of (1) providing a plurality of different mutants of the microorganism; (2) contacting the plurality of mutant microorganisms with antibodies from an animal which has raised an immune response to the microorganism or a part thereof, under conditions whereby if the antibodies bind to the mutant microorganism the mutant microorganism is killed; (3) selecting surviving mutant microorganisms from step (2); (4) identifying the gene containing the mutation in any surviving mutant microorganism; and (5) identifying the polypeptide encoded by the gene. It will be appreciated that by the way in which the polypeptides have been identified, they are highly relevant as antigenic polypeptides.

As described in more detail in the Examples, particular genes identified by the GSI method are the NMB0377, NMB0264, NMB1333, NMB1036, NMB1176, NMB1359 and NMB1138 genes of Neisseria meningitidis. The genome sequence for N. meningitidis is available, for example from The Institute of Genome Research (TIGR); www.tigr.org.

Although these genes form part of the genome that has been sequenced, as far as the inventors are aware, they have not been isolated, the polypeptides they encode have not been produced (and have not been isolated), and there is no indication that the polypeptides they encode may be useful as a component of a vaccine.

Thus, the invention includes the isolated genes as above and in the Examples and variants and fragments and fusions of such variants and fragments, and includes the polypeptides that the genes encode as described above, along with variants and fragment thereof, and fusions of such fragments and variants. Variants, fragments and fusions are described in more detail below. Preferably, the variants, fragments and fusions of the given genes above are ones which encode a polypeptide which gives rise to neutralizing antibodies against N. meningitidis. Similarly, preferably, the variants, fragments and fusions of the polypeptide whose sequence is given above are ones which gives rise to neutralizing antibodies against N. meningitidis. The neutralising antibodies may be produced in any animal with an immune system, for example a rat, mouse or rabbit. The invention also includes isolated polynucleotides encoding the polypeptides whose sequences are given in the Example (preferably the isolated coding region) or encoding the variants, fragments or fusions. The invention also includes expression vectors comprising such polynucleotides and host cells comprising such polynucleotides and vectors (as is described in more detail below). The polypeptides described in the Examples are antigens identified by the method of the invention.

Molecular biological methods for use in the practice of the method of the invention are well known in the art, for example from Sambrook & Russell (2001) Molecular Cloning, a laboratory manual, third edition, Cold Spring Harbor laboratory Press, Cold Spring Harbor, N.Y., incorporated herein by reference.

Variants of the gene may be made, for example by identifying related genes in other microorganisms or in other strains of the microorganism, and cloning, isolating or synthesizing the gene. Typically, variants of the gene are ones which have at least 70% sequence identity, more preferably at least 85% sequence identity, most preferably at least 95% sequence identity with the genes as given above. Of course, replacements, deletions and insertions may be tolerated. The degree of similarity between one nucleic acid sequence and another can be determined using the GAP program of the University of Wisconsin Computer Group.

Variants of the gene are also ones which hybridise under stringent conditions to the gene. By “stringent” we mean that the gene hybridises to the probe when the gene is immobilised on a membrane and the probe (which, in this case is >200 nucleotides in length) is in solution and the immobilised gene/hybridised probe is washed in 0.1×SSC at 65° C. for 10 min. SSC is 0.15 M NaCl/0.015 M Na citrate.

Fragments of the gene (or the variant gene) may be made which are, for example, 20% or 30% or 40% or 50% or 60% or 70% or 80% or 90% of the total of the gene. Preferred fragments include all or part of the coding sequence. The variant and fragments may be fused to other, unrelated, polynucleotides.

The polynucleotide encodes a polypeptide which is immunogenic and is reactive with the antibodies from an animal which has been subjected to the microorganism from which the gene was identified.

The antigen may be the polypeptide as encoded by the gene identified above, and the sequence of the polypeptide may readily be deduced from the gene sequence. In further embodiments, the antigen may be a fragment of the identified polypeptide or may be a variant of the identified polypeptide or may be a fusion of the polypeptide or fragment or variant.

Thus, a particular aspect of the invention provides a polypeptide comprising the amino acid sequence selected from any one of SEQ ID Nos 2, 4, 6, 8, 10, 12, 14; or a fragment or variant thereof or a fusion of such a fragment or variant. Thus, the invention provides the following isolated proteins, or fragments or variants thereof, or fusion of these: NMB1333, NMB0377, NMB0264, NMB1036, NMB1176, NMB1359 and NMB1138 as described below.

Fragments of the identified polypeptide may be made which are, for example, 20% or 30% or 40% or 50% or 60% or 70% or 80% or 90% of the total of the polypeptide. Typically, fragments are at least 10, 15, 20, 30, 40, 50, 100 or more amino acids, but less than 500, 400, 300 or 200 amino acids. Variants of the polypeptide may be made. By “variants” we include insertions, deletions and substitutions, either conservative or non-conservative, where such changes do not substantially alter the normal function of the protein. By “conservative substitutions” is intended combinations such as Gly, Ala; Val, Ile, Leu; Asp, Glu; Asn, Gln; Ser, Thr; Lys, Arg; and Phe, Tyr. Such variants may be made using the well known methods of protein engineering and site-directed mutagenesis.

A particular class of variants are those encoded by variant genes as discussed above, for example from related microorganisms or other strains of the microorganism. Typically the variant polypeptides have at least 70% sequence identity, more preferably at least 85% sequence identity, most preferably at least 95% sequence identity with the polypeptide identified using the method of the invention.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al., (1994) Nucleic Acids Res 22, 4673-80). The parameters used may be as follows:

Fast pairwise alignment parameters: K-tuple (word) size; 1, window size; 5, gap penalty; 3, number of top diagonals; 5. Scoring method: x percent.
Multiple alignment parameters: gap open penalty; 10, gap extension penalty; 0.05. Scoring matrix: BLOSUM.

The fusions may be fusions with any suitable polypeptide. Typically, the polypeptide is one which is able to enhance the immune response to the polypeptide it is fused to. The fusion partner may be a polypeptide that facilitates purification, for example by constituting a binding site for a moiety that can be immobilised in, for example, an affinity chromatography column. Thus, the fusion partner may comprise oligo-histidine or other amino acids which bind to cobalt or nickel ions. It may also be an epitope for a monoclonal antibody such as a Myc epitope.

As discussed above, the variant polypeptides or polypeptide fragments, or fusions of these, are typically ones which give rise to neutralizing antibodies against N. meningitidis.

The invention also includes, therefore, a method of making an antigen as described above, and antigens obtainable or obtained by the method.

The polynucleotides of the invention may be cloned into vectors, such as expression vectors, as is well known on the art. Such vectors maybe present in host cells, such as bacterial, yeast, mammalian and insect host cells. The antigens of the invention may readily be expressed from polynucleotides in a suitable host cell, and isolated therefrom for use in a vaccine.

Typical expression systems include the commercially available pET expression vector series and E. coli host cells such as BL21. The polypeptides expressed may be purified by any method known in the art. Conveniently, the antigen is fused to a fusion partner that binds to an affinity column as discussed above, and the fusion is purified using the affinity column (eg such as a nickel or cobalt affinity column).

It will be appreciated that the antigen or a polynucleotide encoding the antigen (such as a DNA molecule) is particularly suited for use as in a vaccine. In that case, the antigen is purified from the host cell it is produced in (or if produced by peptide synthesis purified from any contaminants of the synthesis). Typically the antigen contains less that 5% of contaminating material, preferably less than 2%, 1%, 0.5%, 0.1%, 0.01%, before it is formulated for use in a vaccine. The antigen desirably is substantially pyrogen free. Thus, the invention further includes a vaccine comprising the antigen, and method for making a vaccine comprising combining the antigen with a suitable carrier, such as phosphate buffered saline. Whilst it is possible for an antigen of the invention to be administered alone, it is preferable to present it as a pharmaceutical formulation, together with one or more acceptable carriers. The carrier(s) must be □acceptable□ in the sense of being compatible with the antigen of the invention and not deleterious to the recipients thereof. Typically, the carriers will be water or saline which will be sterile and pyrogen free.

The vaccine may also conveniently include an adjuvant. Active immunisation of the patient is preferred. In this approach, one or more antigens are prepared in an immunogenic formulation containing suitable adjuvants and carriers and administered to the patient in known ways. Suitable adjuvants include Freund's complete or incomplete adjuvant, muramyl dipeptide, the □Iscoms□ of EP 109 942, EP 180 564 and EP 231 039, aluminium hydroxide, saponin, DEAE-dextran, neutral oils (such as miglyol), vegetable oils (such as arachis oil), liposomes, Pluronic polyols or the Ribi adjuvant system (see, for example GB-A-2 189 141). □Pluronic□ is a Registered Trade Mark. The patient to be immunised is a patient requiring to be protected from infection with the microorganism.

The invention also includes a pharmaceutical composition comprising a polypeptide of the invention or variant or fragment thereof, or fusion of these, or a polynucleotide of the invention or a variant or fragment thereof or fusion of these, and a pharmaceutically acceptable carrier as discussed above.

The aforementioned antigens of the invention (or polynucleotides encoding such antigens) or a formulation thereof may be administered by any conventional method including oral and parenteral (eg subcutaneous or intramuscular) injection. The treatment may consist of a single dose or a plurality of doses over a period of time.

It will be appreciated that the vaccine of the invention, depending on its antigen component (or polynucleotide), may be useful in the fields of human medicine and veterinary medicine.

Diseases caused by microorganisms are known in many animals, such as domestic animals. The vaccines of the invention, when containing an appropriate antigen or polynucleotide encoding an antigen, are useful in man but also in, for example, cows, sheep, pigs, horses, dogs and cats, and in poultry such as chickens, turkeys, ducks and geese.

Thus, the invention also includes a method of vaccinating an individual against a microorganism, the method comprising administering to the individual an antigen (or polynucleotide encoding an antigen) or vaccine as described above. The invention also includes the use of the antigen (or polynucleotide encoding an antigen) as described above in the manufacture of a vaccine for vaccinating an individual.

The antigen of the invention may be used as the sole antigen in a vaccine or it may be used in combination with other antigens whether directed at the same or different disease microorganisms. In relation to N. meningitidis, the antigen obtained which is reactive against NmB may be combined with components used in vaccines for the A and/or C serogroups. It may also conveniently be combined antigenic components which provide protection against Haemophilus and/or Streptococcus pneumoniae. The additional antigenic components may be polypeptides or they may be other antigenic components such as a polysaccharide. Polysaccharides may also be used to enhance the immune response (see, for example, Makela et al (2002) Expert Rev. Vaccines 1, 399-410).

It is particularly preferred in the above vaccines and methods of vaccination if the antigen is the polypeptide encoded by any of the genes as described above (and in the Examples), or a variant or fragment or fusion as described above (or a polynucleotide encoding said antigen), and that the disease to be vaccinated against is Neisseria meningitidis infection (meningococcal disease).

The invention will now be described in greater detail by reference to the following non-limiting Examples.

### EXAMPLE 1

### Genetic Screening for Immunogens (GSI) in N. meningitidis

The application of GSI in this example involves screening libraries of insertional mutants of N. meningitidis for strains which are less susceptible to killing by bactericidal antibodies. GSI is described in more detail in PCT/GB2005/005441 (published as WO 2005/060995 on 7 Jul. 2005).

We have demonstrated the effectiveness of GSI by screening a library of mutants of the sequenced NmB isolate, MC58, with sera raised in mice against a capsule minus of the same strain. A total of 40,000 mutants was analysed with sera raised in mice by intraperitoneal immunisation with the homologous strain; the SBA of this sera is around 2,000 against the wild-type strain. Surviving mutants were detected when the library was exposed to serum at a 1:560 dilution (which kills all wild-type bacteria). To establish whether the transposon insertion in the surviving mutants was responsible for the ability to withstand killing, the mutations were backcrossed into the parental strain, and the backcrossed mutants were confirmed as being more resistant to killing than the wild-type in the SBA. The sequence of the gene affected by the transposon was examined by isolating the transposon insertion site by marker rescue. We found that two of the genes affected were TspA and NMB0338. TspA is a surface antigen which elicits strong CD4+ T cell responses and is recognized by sera from patients (Kizil et al (1999) Infect Immun. 67, 3533-41). NMB0338 is a gene of previously unknown function which encodes a polypeptide that is predicted to contain two transmembrane domains, and is located at the cell surface. The amino acid sequence encoded by NMB0338 is:

(SEQ ID No 15)

MERNGVFGKIVGNRILRMSSEHAAASYPKPCKSFKLAQSWFRVRSCLGGV
FIYGANMKLIYTVIKIIILLLFLLLAVINTDAVTFSYLPGQKFDLPLIVV
LFGAFVVGIIFGMFALFGRLLSLRGENGRLRAEVKKNARLTGKELTAPPA
QNAPESTKQP

There are several practical advantages of using NmB for GSI aside from the public health imperative: a) the bacterium is genetically tractable; b) killing of the bacterium by effector immune mechanism is straightforward to assay; c) the genome sequences are available for three isolates of different serogroups and clonal lineages (IV-A, ET-5, and ET-37 for serogroups A, B, and C, respectively); and d) well-characterised clinical resources are available for this work.

GSI has two potential limitations. First, targets of bactericidal antibodies may be essential. This is unlikely as all known targets of bactericidal antibodies in NmB are non-essential, and no currently licensed bacterial vaccine targets an essential gene product. Second, sera will contain antibodies to multiple antigens, and, loss of a single antigen may not affect the survival of mutants. We have already shown that even during selection with sera raised against the homologus strain, relevant antigens were still identified using appropriate dilutions of sera.

The major advantages of GSI are that 1) the high throughput steps do not involve technically demanding or costly procedures (such as protein expression/purification and immunisation), and 2) human samples can be used in the assay rather than relying solely on animal data. GSI will rapidly pinpoint the subset of surface proteins that elicit bactericidal activity, allowing more detailed analysis of a smaller number of candidates.

### 1. Identification of Targets of Bactericidal Antibodies Using GSI

Murine sera raised against heterologous strains, and human sera, are used to identify cross-reactive antigens. The sera are obtained from:
i) mice immunised by the systemic route with heterologous strains: the strains will be selected and/or constructed to avoid isolates with the same immunotype and sub-serotype.
ii) acute and convalescent sera from patients infected with known isolates of N. meningitidis (provided by Dr R. Wall, Northwick Park)
iii) pre- and post-immunisation samples (provided by the Meningococcal Reference Laboratory) from volunteers receiving defined outer membrane vesicle (OMVs) vaccines derived from the NmB isolate, H44/76.

Each of these sources of sera has specific advantages and disadvantages.

| Serum | | |
|---|---|---|
| source | Advantages | Disadvantages |
| Murine | 1) Defined antigenic exposure. | 1) Animal source |
| | 2) Use of genetically modified | of material |
| | strains to generate immune | |
| | response. | |
| | 3) Naïve samples available | |
| | 4) Examine individuals responses | |
| Patient | 1) Human material | 1) Background |
| sera | 2) Known strain exposure | immunity |
| | 3) Acute and convalescent | 2) Limited material |
| | sera available | |
| Sera | 1) Human material | 1) Background |
| following | 2) Defined antigenic exposure | immunity |
| immunisation | 3) Pre and post immunisation | 2) Limited material |
| with H4476 | sera available | |
| OMVs | 4) Examine individuals responses | |

a) Sera from animals immunised with heterologous strains (ie the sequenced serogroup A or C strains) are used in GSI to select the library of MC58 mutants. We have shown that immunisation with live, attenuated NmB elicits cross-reactive bactericidal antibody responses against serogroup A and C strains. The antigen absent in mutants with enhanced survival in the face of human sera are identified by marker rescue of the disrupted gene.
b) Mutations are identified that confer resistance against killing by heterologous sera, and it is determined whether the gene product is also a target for killing of the sequenced, serogroup A and C strains, Z2491 and FAM18 respectively. The genome databases are inspected for homologues of the genes. If a homologue is present, the transposon insertion is amplified from the MC58 mutant and introduced into the serogroup A and C strains by transformation. The relative survival of the mutant and wild-type strain of each serogroup are compared. Thus, GSI can quickly give information whether the targets of bactericidal activity are conserved and accessible in diverse strains of N. meningitidis, irrespective of serogroup, immunotype and subserotype.
c) Mutants with enhanced survival against sera raised in mice are tested using human sera from either convalescent patients or vaccinees receiving heterologous OMV vaccines (derived from H44/76). This addresses the important question of whether the targets are capable of eliciting bactericidal antibodies in human. With other vaccine approaches, this information is only gained at the late, expensive stage of clinical trials that requires GMP manufacture of vaccine candidates.

The advantages are that GSI is a high-throughput analysis performed using simple, available techniques. Antigens which elicit bactericidal antibodies in humans and which mediate killing of multiple strains can be identified rapidly as GSI is flexible with respect to the bacterial strain and sera used. Mutants selected using human sera are analysed in the same way as those selected by murine sera.

### 2. Assessment of the Antibody Response of Recombinant GSI Antigens

Proteins which are targets of bactericidal antibodies that are recognised by sera from convalescent patients and vaccines are expressed in E. coli using commercially available vectors. The corresponding open reading frames are amplified by PCR from MC58, and ligated into vectors such as pCR Topo CT or pBAD/His, to allow protein expression under the control of a T7 or arabinose-inducible promoter, respectively. Purification of the recombinant proteins from total cellular protein is performed via the His Tag fused to the C terminus of the protein on a Nickel or Cobalt column.

Adult New Zealand White rabbits are immunized on two occasions separated by four weeks by subcutaneous injection with 25 îg of purified protein with Freund's incomplete adjuvant. Sera from animals will be checked prior to immunisation for pre-existing anti-Nm antibodies by whole cell ELISA. Animals which have an initial serum titre of <1:2 are used for immunisation experiments. Post-immunisation serum are obtained two weeks after the second immunisation. To confirm that specific antibodies have been raised, pre- and post-immunisation serum is tested by i) Western analysis against the purified protein and ii) ELISA using cells from the wild-type and the corresponding mutant (generated by GSI).

SBAs will be performed against MC58 (the homologous strain), and the sequenced serogroup A and C strains with the rabbit immune serum. The assay will be performed in triplicate on at least two occasions. SBAs of >8 will be considered significant. The results provide evidence of whether the protein candidates can elicit bactericidal antibodies as recombinant proteins.

### 3. Establishing the Protective Efficacy of GSI Antigens

All the candidates are tested for their ability to protect animals against live bacterial challenge as this allows any aspect of immunity (cellular or humoral) to be assessed in a single assay. We have established a model of active immunisation and protection against live bacterial infection. In this model, adult mice are immunised on days 0 and 21, and on day 28 receive live bacterial challenge of 10⁶ or 10⁷ CFU of MC58 intraperitoneally in iron dextran (as the supplemental iron source). The model is similar to that described for evaluation of the protective efficacy of immunisation with Tbps Danve et al (1993) Vaccine 11, 1214-1220. Non-immunised animals develop bacteraemia within 4 hours of infection, and show signs of systemic illness by 24 hours. We have already been able to demonstrate the protective efficacy of both attenuated Nm strains and a protein antigen against live meningococcal challenge; PorA is an outer membrane protein that elicits bactericidal antibodies, but which is not a lead vaccine candidate because of extensive antigenic variation (Bart et al (1999) Infect Immun. 67, 3832-3846.

Six week old, BALB/c mice (group size, 35 animals) receive 25 îg of recombinant protein with Freund's incomplete adjuvant subcutaneously on days day 0 and 21, then are challenged with 10⁶ (15 animals) or 10⁷ (15 animals) CFU of MC58 intraperitoneally on day 28. Two challenge doses are used to examine the vaccine efficacy at a high and low challenge dose; sera are obtained on day 28 from the remaining five animals in each group, and from five animals before the first immunisation and stored at −70° C. for further immunological assays. Animals in control groups receive either i) adjuvant alone, ii) recombinant refolded PorA, and iii) a live, attenuated Nm strain. To reduce the overall number of animals in control groups, sets of five candidates will be tested at one time (number of groups=5 candidates+3 controls). Survival of animals in the groups is compared by Mann Whitney U Test. With group sizes of 15 mice/dose, the experiments are powered to show a 25% difference in survival between groups.

For vaccines which show significant protection against challenge, a repeat experiment is performed to confirm the finding. Furthermore, to establish that vaccination with a candidate also elicits protection against bacteraemia, levels of bacteraemia are determined during the second experiment; blood is sampled at 22 hr post-infection in immunised and un-immunised animals (bacteraemia is maximal at this time). The results are analysed using a two-tailed Student-T test to determine if there is a significant reduction in bacteraemia in vaccinated animals.

### Further Materials and Methods Used

Mutagenesis of Neisseria meningitidis

For work with Neisseria meningitidis, mutants were constructed by in vitro mutagenesis. Genomic DNA from N. meningitidis was subjected to mutagenesis with a Tn5 derivative containing a marker encoding resistance to kanamycin, and an origin of replication which is functional in E. coli. These elements are bound by composite Tn5 ends. Transposition reactions were carried out with a hyperactive variant of Tn5 and the DNA repaired with T4 DNA polymerase and ligase in the presence of ATP and nucleotides. The repaired DNA was used to transform N. meningitidis to kanamycin resistance. Southern analysis confirmed that each mutant contained a single insertion of the transposon only.

### Serum Bactericidal Assays (SBAs)

Bacteria were grown overnight on solid media (brain heart infusion media with Levanthals supplement) and then re-streaked to solid media for four hours on the morning of experiments. After this time, bacteria were harvested into phosphate buffered saline and enumerated. SBAs were performed in a 1 ml volume, containing a complement source (baby rabbit or human) and approximately 10⁵ colony forming units. The bacteria were collected at the end of the incubation and plated to solid media to recover surviving bacteria.

### Isolating the Transposon Insertion Sites

Genomic DNA will be recovered from mutants of interest by standard methods and digested with PvuII, EcoRV, and DraI for three hours, then purified by phenol extraction. The DNA will then be self-ligated in a 100 microlitre volume overnight at 16° C. in the presence of T4 DNA ligase, precipitated, then used to transform E. coli to kanamycin resistance by electroporation.

### EXAMPLE 2

### Further Screening and Results Thereof

GSI has been used to screen a library of approximately 40,000 insertional mutants of MC58. The library was constructed by in vitro Tn5 mutagenesis, using a transposon harbouring the origin of replication from pACYC 184.

MC58 was chosen as it is a serogroup β isolate of N. meningitidis, and the complete genome sequence of this strain is known.

The library is always screened in parallel with the wild-type strain as a control, and the number of colonies recovered from the library and the wild-type is shown.

The following additional antigens were identified using essentially the methodology described above:

NMB1333 Nucleic acid sequence
ATGCGCTACAAACCCCTTCTGCTTGCCCTGATGCTCGTTTTTTCCACGCCCGCCGTTGCC
GCCCACGACGCGGCACACAACCGTTCCGCCGAAGTGAAAAAACAGACGAAGAACAAAAAA
GAACAGCCCGAAGCGGCGGAAGGCAAAAAAGAAAAAGGCAAAAATGGCGCAGTGAAAGAT
AAAAAAACAGGCGGCAAAGAGGCGGCAAAAGAGGGCAAAGAGTCCAAAAAAACCGCCAAA
AACCGCAAAGAAGCAGAGAAGGAGGCGACATCCAGGCAGTCTGCGCGCAAAGGACGCGAA
GGGGATAAGAAATCGAAGGCGGAACACAAAAAGGCACATGGCAAGCCCGTGTCCGGATCC
AAAGAAAAAAACGCAAAAACACAGCCTGAAAACAAACAAGGCAAAAAAGAGGCAAAAGGA
CAGGGCAATCCGCGCAAGGGCGGCAAGGCGGAAAAAGACACTGTTTCTGCAAATAAAAAA
GTCCGTTCCGACAAGAACGGCAAAGCAGTGAAACAGGACAAAAAATACAGGGAAGAGAAA
AATGCCAAAACCGATTCCGACGAATTGAAAGCCGCCGTTGCCGCTGCCACCAATGATGTC
GAAAACAAAAAAGCCCTGCTCAAACAAAGCGAAGGAATGCTGCTTCATGTCAGCAATTCC
CTCAAACAGCTTCAGGAAGAGCGTATCCGCCAAGAGCGTATCCGTCAGGCGCGCGGCAAC
CTTGCTTCCGTCAACCGCAAACAGCGCGAGGCTTGGGACAAGTTCCAAAAACTCAATACC
GAGCTGAACCGTTTGAAAACGGAAGTCGCCGCTACGAAAGCGCAGATTTCCCGTTTCGTA
TCGGGGAACTATAAAAACAGCCAGCCGAATGCGGTTGCCCTGTTCCTGAAAAACGCCGAA
CCGGGTCAGAAAAACCGCTTTTTGCGTTATACGCGTTATGTAAACGCCTCCAATCGGGAA
GTTGTCAAGGATTTGGAAAAACAGCAGAAGGCTTTGGCGGTACAAGAGCAGAAAATCAAC
AATGAGCTTGCCCGTTTGAAGAAAATTCAGGCAAACGTGCAATCTCTGCTGAAAAAACAG
GGTGTAACCGATGCGGCGGAACAGACGGAAAGCCGCAGACAGAATGCCAAAATCGCCAAA
GATGCCCGAAAACTGCTGGAACAGAAAGGGAACGAGCAGCAGCTGAACAAGCTCTTGAGC
AATTTGGAGAAGAAAAAGGCCGAACACCGCATTCAGGATGCGGAAGCAAAAAGAAAATTG
GCTGAAGCCAGACTGGCGGCAGCCGAAAAAGCCAGAAAAGAAGCGGCGCAGCAGAAGGCT
GAAGCACGACGTGCGGAAATGTCCAACCTGACCCGCGAAGACAGGAACATCCAAGCGCCT
TCGGTTATGGGTATCGGCAGTGCCGACGGTTTCAGCCGCATGCAAGGACGTTTGAAAAAA
CCGGTTGACGGTGTGCCGACCGGACTTTTCGGGCAGAACCGGAGCGGCGGCGATATTTGG
AAAGGCGTGTTCTATTCCACTGCACCGGCAACGGTTGAAAGCATTGCGCCGGGAACGGTA
AGCTATGCGGACGAGTTGGACGGCTACGGCAAAGTGGTCGTGGTCGATCACGGCGAGAAC
TACATCAGCATCTATGCCGGTTTGAGCGAAATTTCCGTCGGCAAGGGTTATATGGTCGCG
GCAGGAAGCAAAATCGGCTCGAGCGGGTCGCTGCGGGACGGGGAAGAGGGGCTTTACCTG
CAAATACGTTATCAAGGTCAGGTATTGAACCCTTCGAGCTGGATACGTTGA
NMB1333 Amino acid sequence
MRYKPLLLALMLVFSTPAVAAHDAAHNRSAEVKKQTKNKKEQPEAAEGKKEKGKNGAVKD
KKTGGKEAAKEGKESKKTAKNRKEAEKEATSRQSARKGREGDKKSKAEHKKAHGKPVSGS
KEKNAKTQPENKQGKKEAKGQGNPRKGGKAEKDTVSANKKVRSDKNGKAVKQDKKYREEK
NAKTDSDELKAAVAAATNDVENKKALLKQSEGMLLHVSNSLKQLQEERIRQERIRQARGN
LASVNRKQREAWDKFQKLNTELNRLKTEVAATKAQISRFVSGNYKNSQPNAVALFLKNAE
PGQKNRFLRYTRYVNASNREVVKDLEKQQKALAVQEQKINNELARLKKIQANVQSLLKKQ
GVTDAAEQTESRRQNAKIAKDARKLLEQKGNEQQLNKLLSNLEKKKAEHRIQDAEAKRKL
AEARLAAAEKARKEAAQQKAEARRAEMSNLTAEDRNIQAPSVMGIGSADGFSRMQGRLKK
PVDGVPTGLFGQNRSGGDIWKGVFYSTAPATVESIAPGTVSYADELDGYGKVVVVDHGEN
YISIYAGLSETSVGKGYMVAAGSKTGSSGSLPDGEEGLYLQIRYQGQVLNPSSWIR
NMB0377 Nucleic acid sequence
ATGGCGTTTTGCACCAGTTTGGGAGTGATGATGGAAACACAGCTTTACATCGGCATCATG
TCGGGAACCAGCATGGACGGGGCGGATGCCGTACTGATACGGATGGACGGCGGCAAATGG
CTGGGCGCGGAAGGGCACGCCTTTACCCCCTACCCCGGCAGGTTACGCCGCCAATTGCTG
GATTTGCAGGACACAGGCGCAGACGAACTGCACCGCAGCAGGATTTTGTCGCAAGAACTC
AGCCGCCTATATGCGCAAACCGCCGCCGAACTGCTGTGCAGTCAAAACCTCGCACCGTCC
GACATTACCGCCCTCGGCTGCCACGGGCAAACCGTCCGACACGCGCCGGAACACGGTTAC
AGCATACAGCTTGCCGATTTGCCGCTGCTGGCGGAACGGACGCGGATTTTTACCGTCGGC
GACTTCCGCAGCCGCGACCTTGCGGCCGGCGGACAAGGCGCGCCACTCGTCCCCGCCTTT
CACGAAGCCCTGTTCCGCGACAACAGGGAAACACGCGCGGTACTGAACATCGGCGGGATT
GCCAACATCAGCGTACTCCCCCCCGACGCACCCGCCTTCGGCTTCGACACAGGGCCGGGC
AATATGCTGATGGACGCGTGGACGCAGGCACACTGGCAGCTTCCTTACGACAAAAACGGT
GCAAAGGCGGCACAAGGCAACATATTGCCGCAACTGCTCGACAGGCTGCTCGCCCACCCG
TATTTCGCACAACCCCACCCTAAAAGCACGGGGCGCGAACTGTTTGCCCTAAATTGGCTC
GAAACCTACCTTGACGGCGGCGAAAACCGATACGACGTATTGCGGACGCTTTCCCGTTTT
ACCGCGCAAACCGTTTGCGACGCCGTCTCACACGCAGCGGCAGATGCCCGTCAAATGTAC
ATTTGCGGCGGCGGCATCCGCAATCCTGTTTTAATGGCGGATTTGGCAGAATGTTTCGGC
ACACGCGTTTCCCTGCACAGCACCGCCGACCTGAACCTCGATCCGCAATGGGTGGAAGCC
GCCGCATTTGCGTGGTTGGCGGCGTGTTGGATTAATCGCATTCCCGGTAGTCCGCACAAA
GCAACCGGCGCATCCAAACCGTGTATTCTGGGCGCGGGATATTATTATTGA
NMB0377 Amino acid sequence
MAFCISLGVMMETQLYIGIMSGTSMDGADAVLIRMDGGKWLGAEGHAFTPYPGRLRRQLL
DLQDTGADELHRSRILSQELSRLYAQTAAELLCSQNLAPSDITALGCHGQTVRHAPEHGY
SIQLADLPLLAERTRIFTVGDFRSRDLAAGGQGAPLVPAFHEALFRDNRETRAVLNIGGI
ANISVLPPDAPAFGFDTGPGNMLMDAWTQAHWQLPYDKNGAKAAQGNILPQLLDRLLAHP
YFAQPHPKSTGRELFALNWLETYLDGGENRYDVLRTLSRFTAQTVCDAVSHAAADARQMY
ICGGGIRNPVLMADLAECFGTRVSLHSTADLNLDPQWVEAAAFAWLAACWINRIPGSPHK
ATGASKPCILGAGYYY
NMB0264 Nucleic acid sequence
ATGTTGAACAAAATATTTTCCTGGTTCGAGTCCCGAATCGACCCTTATCCCGAAGCCGCC
CCGAAAACGCCAGAAAAAGGCTTGTGGCGGTTTGTCTGGAGCAGCATGGCCGGCGTGCGG
AAATGGATAGCCGCCCTGGCTGCGCTGACCGCCGGCATCGGCATTATGGAAGCCCTGGTT
TTTCAATTTATGGGCAAAATCGTGGAGTGGCTCGGCAAATACGCGCCCGCCGAACTGTTT
GCCGAAAAAAGTTGGGAACTGGCGGCAATGGCGGCGATGATGGTATTTTCGGTTGCGTGG
GCGTTTGCCGCGTCCAACGTGCGCCTGCAAACCCTTCAGGGCGTGTTCCCCATGCGCCTG
CGCTGGAACTTCCACCGCCTGATGCTGAACCAAAGCCTCGGTTTTTATCAGGACGAATTT
GCCGGACGCGTGTCCGCCAAAGTCATGCAGACCGCGCTGGCGTTGCGCGACGCGGTGATG
ACGGTTGCCGATATGGTCGTTTATGTGTCGGTGTATTTCATTACCTCCGGCGTGATTCTC
GCCTCGCTCGACTCATGGCTGCTGCTGCCCTTTATCGGCTGGATTGTCGGTTTCGCTTCG
GTGATGCGCCTGCTGATTCCCAAATTGGGGCAAACCGCCGCATGGCAGGCGGATGCCCGC
TCGCTGATGACCGGCCGCATTACCGATGCCTATTCCAATATCGCCACCGTCAAACTCTTC
TCCCACGGCGCGCGTGAAGCCGCCTATGCCAAGCAGTCGATGGAAGAATTTATGGTTACG
GTGCGCGCCCAAATGCGGCTGGCGACGCTGCTGCATTCGTGCAGCTTCATCGTCAACACC
TCCCTGACCCTCTCCACCGCCGCACTGGGCATCTGGCTCTGGCACAACGGGCAGGTCGGC
GTGGGCGCGGTTGCTACAGCCACCGCCATGGCGTTGGCCGTCAACGGTTTGTCGCAATAC
ATTATGTGGGAATCCGCGCGGCTGTTTGAAAACATCGGCACCGTCGGCGACGGCATGGCA
ACCCTGTCCAAACCGCACACCATCCTCGACAAGCCCCGGGCACTGCCGCTGAACGTGCCG
CAAGGCGCAATCAAATTTGAACACGTCGATTTCTCCTACGAAGCGGGCAAACCGCTGCTC
AACGGCTTCAACCTCACCATCCGCCCGGGCGAAAAAGTCGGCTTGATCGGACGCAGCGGC
GCGGGCAAATCCACCATCGTCAACCTGCTTTTGCGCTTCTACGAACCGCAAAGCGGCACG
GTTTCGATCGACGGGCAGGACATAAGCGGCGTTACCCAAGAATCTTTACGCGCCCAAATC
GGTTTGGTCACGCAAGATACCTCGCTGCTGCACCGTTCCGTGCGCGACAACATTATTTAC
GGCCGCCCCGACGCGACCGATGCCGAAATGGTTTCTGCCGCCGAACGCGCCGAAGCCGCC
GGCTTCATCCCCGACCTTTCCGATGCCAAAGGGCGGCGCGGCTACGACGCACACGTCGGC
GAACGCGGCGTGAAACTCTCCGGCGGGCAACGCCAGCGCATCGCCATCGCCCGCGTGATG
CTCAAAGACGCACCGATTCTTCTTTTGGACGAAGCCACCAGCGCGCTCGATTCCGAAGTC
GAAGCCGCCATCCAAGAAAGCCTCGACAAAATGATGGACGGCAAAACCGTCATCGCCATC
GCCCACCGCCTCTCCACCATCGCCGCAATGGACAGGCTCGTCGTCCTCGACAAAGGCCGC
ATCATCGAAGAAGGCACACACGCCGAACTCCTCGAAAAACGCGGGCTTTACGCCAAACTC
TGGGCGCACCAGAGCGGCGGCTTCCTCAACGAACACGTCGAGTGGCAGCACGACTGA
NMB0264 Amino acid sequence
MLNKIFSWFESRIDPYPEAAPKTPEKGLWRFVWSSMAGVRKWIAALAALTAGIGIMEALV
FQFMGKIVEWLGKYAPAELFAEKSWELAAMAAMMVFSVAWAFAASNVRLQTLQGVFPMRL
RWNFHRLMLNQSLGFYQDEFAGRVSAKVMQTALALRDAVMTVADMVVYVSVYFITSGVIL
ASLDSWLLLPFIGWIVGFASVMRLLIPKLGQTAAWQADARSLMTGRITDAYSNIATVKLF
SHGAREAAYAKQSMEEFMVTVRAQMRLATLLHSCSFIVNTSLTLSTAALGIWLWHNGQVG
VGAVATATAMALRVNGLSQYIMWESARLFENIGTVGDGMATLSKPHTILDKPRALPLNVP
QGAIKFEHVDFSYEAGKPLLNGFNLTIRPGEKVGLIGRSGAGKSTIVNLLLRFYEPQSGT
VSIDGQDISGVTQESLRAQIGLVTQDTSLLHRSVRDNIIYGRPDATDAEMVSAAERAEAA
GFIPDLSDAKGRRGYDAHVGERGVKLSGGQRQRIAIARVMLKDAPILLLDEATSALDSEV
EAAIQESLDKMMDGKTVIAIAHRLSTIAAMDRLVVLDKGRIIEEGTHAELLEKRGLYAKL
WAHQSGGFLNEHVEWQHD
NMB1036 Nucleic acid sequence
ATGACAGCACAAACCCTCTACGACAAACTTTGGAACAGCCACGTCGTCCGCGAAGAAGAA
GACGGCACCGTCCTGCTCTACATGOACCGCCATTTGGTGCACGAAGTTACCAGCCCTCAG
GCATTTGAAGGCTTGAAAATGGCGGGGCGCAAGCTGTGGCGCATCGACAGCGTCGTCTCC
ACCGCCGACCACAACACCCCGACCGGCGATTGGGACAAAGGCATCCAAGACCCGATTTCC
AAGCTGCAAGTCGATACTTTGGACAAAAACATTAAAGAGTTTGGCGCACTCGCCTATTTT
CCGTTTATGGACAAAGGTCAGGGCATCGTACACGTTATGGGCCCCGAACAAGGCGCGACC
CTGCCCGGTATGACCGTCGTCTGCGGCGACTCGCACACTTCCACCCACGGCGCATTCGGC
GCACTGGCGCACGGCATCGGCACTTCCGAAGTCGAGCACACCATGGCGACCCAATGTATT
ACCGCGAAAAAATCCAAATCCATGCTGATTTCCGTTGACGGCAAATTAAAAGCGGGCGTT
ACCGCCAAAGACGTGGCGCTCTACATCATCGGGCAAATCGGCACGGCAGGCGGTACAGGC
TACGCCATCGAGTTTGGCGGCGAAGCCATCCGCAGCCTTTCTATGGAAAGCCGCATGACT
TTATGCAATATGGCGATTGAGGCAGGCGCGCGCTCAGGCATGGTTGCCGTCGACCAAACC
ACCATCGACTACGTAAAAGATAAACCCTTCGCACCCGAAGGCGAAGCGTGGGACAAAGCC
GTCGAGTACTGGCGTACGCTGGTGTCTGACGAAGGTGCGGTATTCGACAAAGAATACCGT
TTCAACGCCGAAGACATCGAACCGCAAGTCACTTGGGGTACCTCGCCTGAAATGGTTTTA
GACATCAGCAGCAAAGTGCCGAATCCTGCCGAAGAAACCGATCCGGTCAAACGCAGCGGT
ATGGAACGCGCCCTTGAATACATGGGCTTGGAAGCCGGTACGCCATTAAACGAAATCCCC
GTCGACATCGTATTCATCGGCTCTTGCACCAACAGCCGCATCGAAGACTTGCGCGAAGCC
GCCGCCATCGCCAAAGACCGCAAAAAAGCCGCCAACGTACAGCGCGTGTTAATCGTCCCC
GGCTCCGGTTTGGTTAAAGAACAAGCCGAAAAAGAAGGCTTGGACAAAATTTTCATCGAA
GCCGGTTTTGAATGGCGCGAACCGGGCTGTTCGATGTGTCTCGCCATGAACGCCGACCGC
CTGACCCCGGGGCAACGCTGCGCCTCCACCTCCAACCGTAACTTTGAAGGCCGTCAAGGC
AACGGCGGACGTACCCACCTCGTCAGCCCCGCTATGGCAGCAGCCGCCGCCGTTACCGGC
CGCTTTACCGACATCCGCATGATGGCGTAA
NMB1036 Amino acid sequence
MTAQTLYDKLWNSHVVREEEDGTVLLYIDRHLVHEVTSPQAFEGLKMAGRKLWRIDSVVS
TADHNTPTGDWDKGIQDPISKLQVDTLDKNIKEFGALAYFPFMDKGQGIVHVMGPEQGAT
LPGMTVVCGDSHTSTHGAFGALAHGIGTSEVEHTMATQCITAKKSKSMLISVDGKLKAGV
TAKDVALYIIGQIGTAGGTGYAIEFGGEAIRSLSMESRMTLCNMAIEAGARSGMVAVDQT
TIDYVKDKPFAPEGEAWDKAVEYWRTLVSDEGAVFDKEYRFNAEDIEPQVTWGTSPEMVL
DISSKVPNPAEETDPVKRSGMERALEYMGLEAGTPLNEIPVDIVFIGSCTNSRIEDLREA
AAIAKDRKKAANVQRVLIVPGSGLVKEQAEKEGLDKIFIEAGFEWREPGCSMCLAMNADR
LTPGQRCASTSNRNFEGRQGNGGRTELVSPAMAAAAAVTGRFTDIRMMA
NMB1176 Nucleic acid sequence
ATGAAAGACAAGCACGATTCTTCCGCCATGCGGCTGGACAAATGGCTTTGGGCGGCACGT
TTTTTCAAGACCCGTTCCCTTGCGCAAAAGCACATCGAACTGGGTAGGGTTCAAGTAAAC
GGCTCGAAGGTCAAAAACAGTAAAACCATAGACATCGGCGATATTATCGACCTGACGCTC
AATTCCCTTCCCTATAAAATCAAGGTTAAAGGTTTGAACCACCAACGCCGCCCGGCATCC
GAGGCGCGGCTTCTGTATGAAGAGGACGCGAAAACGGCAACATTGAGGGAAGAGCGCAAA
CAGCTCGACCAATTCAGCCGCATCACTTCCGCCTATCCCGACGGCAGACCGACCAAGCGC
GACCGCCGCCAACTGGACAGGCTGAAAAAAGGAGACTGGTAA
NMB1176 Amino acid sequence
MKDKHDSSAMRLDKWLWAARFFKTRSLAQKHIELGRVQVNGSKVKNSKTIDIGDIIDLTL
NSLPYKIKVKGLNEQRRPASEARLLYEEDAKTATLREERKQLDQFSRITSAYPDGRPTKR
DRRQLDRLKKGDW
NMB1359 Nucleic acid sequence
ATGAACCACACCGTTACCCTGCCCGACCAAACCACCTTTGCCGCCAACGACGGCGAAACC
GTTTTGACCGCTGCCGCCCGTCAAAACCTCAACCTGCCCCATTCCTGCAAAAGCGGTGTC
TGCGGACAATGCAAAGCCGAACTGGTCAGCGGCGATATTCAAATGGGCGGACACTCGGAA
CAGGCTTTATCCGAAGCAGAAAAAGCGCAAGGCAAGATTTTGATGTGCTGCACCACTGCG
CAAAGCGATATCAACATCAACATCCCCGGCTACAAAGCCGATGCCCTACCCGTCCGCACC
CGCCCCGCACGCATCGAAAGTATTATTTTCAAACACGATGTCGCCCTCCTGAAACTTGCC
CTGCCCAAAGCCCCGCCGTTTGCCTTCTACGCCGGGCAATACATTGATTTACTGCTGCCG
GGCAACGTCAGCCGCAGCTACTCCATCGCCAATTTACCCGACCAAGAAGGCATTTTGGAA
CTGCACATCCGCAGGCACGAAAACGGTGTCTGCTCGGAAATGATTTTCGGCAGCGAACCC
AAAGTCAAAGAAAAAGGCATCGTCCGCGTTAAAGGCCCGCTCGGTTCGTTTACCTTGCAG
GAAGACAGCGGCAAACCCGTCATCCTGCTGGCAACCGGCACAGGCTACGCCCCCATCCGC
AGCATCCTGCTCGACCTTATCCGCCAAGGCAGCAACCGCGCCGTCCATTTCTACTGGGGC
GCGCGTCATCAGGATGATTTGTATGCCCTCGAAGAAGCACAAGGGTTGGCATGCCGTCTG
AAAAACGCCTGCTTCACCCCCGTATTGTCCCGCCCCGGAGAGGGCTGGCAGGGAAGAAAT
GGTCACGTACAAGACATCGCGGCACAAGACCACCCCGACCTGTCGGAATACGAAGTATTT
GCCTGCGGTTCTCCGGCCATGACCGAACAAACAAAGAATCTGTTTGTGCAACAGCATAAG
CTGCCGGAAAACTTGTTTTTCTCCGACGCATTCACGCCGTCCGCATCATAA
NMB1359 Amino acid sequence
MNHTVTLPDQTTFAANDGETVLTAAARQNLNLPHSCKSGVCGQCKAELVSGDIQMGGHSE
QALSEAEKAQGKILMCCTTAQSDININIPGYKADALPVRTLPARIESIIFKHDVALLKLA
LPKAPPFAFYAGQYIDLLLPGNVSRSYSIANLPDQEGILELHIRRHENGVCSEMIFGSEP
KVKEKGIVRVKGPLGSFTLQEDSGKPVILLATGTGYAPIRSILLDLIRQGSNRAVHFYWG
ARHQDDLYALEEAQGLACRLKNACFTPVLSRPGEGWQGRNGHVQDIAAQDHPDLSEYEVF
ACGSPAMTEQTKNLFVQQHKLPENLFFSDAFTPSAS
NMB1138 Nucleic acid sequence
ATGAAAGACAAGCACGATTCTTCCGCCATGCGGCTGGACAAATGGCTTTGGGCGGCACGT
TTTTTCAAGACCCGTTCCCTTGCGCAAAAGCACATCGAACTGGGTAGGGTTCAAGTAAAC
GGCTCGAAGGTCAAAAACAGTAAAACCATAGACATCGGCGATATTATCGACCTGACGCTC
AATTCCCTTCCCTATAAAATCAAGGTTAAAGGTTTGAACCACCAACGCCGCCCGGCATCC
GAGGCGCGGCTTCTGTATGAAGAGGACGCGAAAACGGCAACATTGAGGGAAGAGCGCAAA
CAGCTCGACCAATTCAGCCGCATCACTTCCGCCTATCCCGACGGCAGACCGACCAAGCGC
GACCGCCGCCAACTGGACAGGCTGAAAAAAGGAGACTGGTAA
NMB1138 Amino acid sequence
MKDKHDSSAMRLDKWLWAARFFKTRSLAQKHIELGRVQVNGSKVKNSKTIDIGDIIDLTL
NSLPYKIKVKGLNHQRRPASEARLLYEEDAKTATLREERKQLDQFSRITSAYPDGRPTKR
DRRQLDRLKKGDW

### Schedule of SEQ ID Nos

!SEQ ID No? Sequence

| | |
|---|---|
| 1 | NMB1333 DNA |
| 2 | NMB1333 Protein |
| 3 | NMB0377 DNA |
| 4 | NMB0377 Protein |
| 5 | NMB0264 DNA |
| 6 | NMB0264 Protein |
| 7 | NMB1036 DNA |
| 8 | NMB1036 Protein |
| 9 | NMB1176 DNA |
| 10 | NMB1176 Protein |
| 11 | NMB1359 DNA |
| 12 | NMB1359 Protein |
| 13 | NMB1138 DNA |
| 14 | NMB1138 Protein |

1. A polypeptide comprising an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, and a fragment or variant thereof or a fusion of such a fragment or variant.

2. A polynucleotide encoding a polypeptide according to claim 1.

3-4. (canceled)

5. The polypeptide of claim 1, wherein said polypeptide is produced recombinantly.

6. The polypeptide according to of claim 1, wherein said polypeptide is chemically synthesized.

7. A method of vaccinating an individual against Neisseria meningitidis, the method comprising administering to the individual a polypeptide according to claim 1.

8. (canceled)

9. A pharmaceutical composition comprising a polypeptide according to claim 1 and a pharmaceutically acceptable carrier.

10. A method of vaccinating an individual against Neisseria meningitidis, the method comprising administering to the individual a polynucleotide according to claim 2.

11. A pharmaceutical composition comprising a polynucleotide according to claim 2 and a pharmaceutically acceptable carrier.
